(19) United States
(12) Patent Application Publication
(10) Pub. No.: US 2004/0220167 A1
(43) Pub. Date: Nov. 4, 2004

(54) Methods of treating neuralgic pain

(76) Inventor: Nasrollah Samiy, Charlotte, NC (US)

Correspondence Address: OSTROLENK FABER GERB & SOFFEN, 1180 AVENUE OF THE AMERICAS, NEW YORK, NY, 100368403

(21) Appl. No.: 10/428,266

(22) Filed: May 2, 2003

(51) Int. Cl. A61K 31/409; A61K 31/555

(52) U.S. Cl. 514/185; 514/410

(57) ABSTRACT

A method of treating pain in a subject is disclosed which consists of administering a pharmaceutically effective amount of a porphyrin compound. In particular, the porphyrin is a green porphyrin and the pain is associated with a nerve injury pain or neuralgia.

### BACKGROUND OF THE INVENTION

1. Field of the Invention

The present invention relates to methods of treating pain, specially neuralgic using porphyrin compounds or derivatives thereof.

2. Description of the Related Art

For many types of pain, the method for treating the pain is through administration of aspirin or any of a number of non-steroidal anti-inflammatory agents, NSAIDs. Frequently, the administration of the NSAID provides the relief sought. However, if the relief of pain is insufficient with NSAIDs alone, these agents may be combined with orally effective morphine-like agents, such as codeine and other opioids. Because these two agents exert their effects by different mechanisms, combinations of these two classes of drugs usually can achieve an analgesic effect that would otherwise require a higher dose of opioid, but with fewer side effects. Nonetheless, even at a lesser dose of opioid, resulting in fewer side effects, it has been well documented that any dose of an opioid has the potential for severe side effects. For example, morphine and its related opioids may cause respiratory depression, nausea, vomiting, dizziness, mental clouding, dysphoria, pruritus, constipation, increased pressure in the biliary tract, urinary retention, hypotension, tolerance, and physical dependence. (The Pharmacological Basis of Therapeutics, 9th edition, Macmillan Publishing Co., 1996, pp 533-540).

Controlling pain associated with neuralgia and more specifically post-herpetic neuralgia is of particular challenge. This is because the mechanism of pain generation in post-herpetic neuralgia is unknown. Post-herpetic neuralgia is the predominant morbidity associated with development of herpes-zoster, also known as shingles. The neuralgia typically lasts for from one to six months and is often excruciatingly painful.

Evidence has accrued which shows that herpes-zoster is caused by reactivation of latent varicella virus (Straus et al., Ann. Int. Med. 1988, 108:221-237; Hyman et al., Lancet 1983, 2:814-816; Gilden et al., Nature 1983, 306:478-80; Croen et al., Proc. Natl. Acad. Sci. USA 1988, 85:9773-9777; Mahalingham at al., New Eng. J. Med. 1990, 323:627-631. The initial varicella infection may have occurred as a result of infantile chickenpox or as a result of immunization with a live-attenuated varicella zoster virus vaccine to prevent chickenpox. In either case, the virus appears to remain in the infected individual's system long after chickenpox or vaccination. The locus of VZV latency appears to be neural cells within dorsal root ganglia.

Years after VZV has become latent, the virus reactivates by an as yet poorly understood mechanism. Nonetheless, the reactivation of VZV and its subsequent replication gives rise to herpes zoster. It is in the course of and subsequent to this reactivation of VZV that severe post-herpetic neuralgia develops.

Post-herpetic neuralgia (PHN) begins with a cutaneous rash and the chronic state is notable for skin scarring and painfully sensitive skin (allodynia). Although the initial outbreak may be widespread, occasionally appearing to cover more than the area of skin innervated by a single dorsal root ganglion, most PHN patients are able to localize a limited area of skin as the source of their pain. PHN patients nearly always have a sensory deficit in the region obtained.

The majority of work carried out on topical agents for analgesia in recent years has been in patients with PHN. Other conditions, particularly diabetic neuropathy, have been treated in clinical trials and clinical practice with topical agents, primarily capsaicin. Topical therapies have represented a very attractive alternative to oral medications for conditions like PHN. The primarily elderly patients with PHN frequently cannot be treated with tricyclic antidepressants-because of pre-existing cognitive impairment, cardiac disease, or systemic illness. Diabetic autonomic dysfunction may significantly enhance orthostatic hypotension from tricyclic antidepressants. Side effects like constipation, dry mouth and sedation may prove so bothersome that compliance becomes a major problem in therapy. Anticonvulsants are of uncertain efficacy in PHN, though carbamazepine and antiarrhythmics like mexiletine are effective for diabetic neuropathy. Non-narcotic analgesics are rarely effective and benzodiazepines have been proven ineffective. Opioids may be effective, but have not been adequately evaluated as long term treatment for PHN or diabetic neuropathy.

The use of local anesthetics to control the pain of herpes-zoster PHN has a history dating back to Wood's 1929 report of complete relief of ophthalmic PHN from injection of procaine into the supraorbital nerve (Wood, Am. J. Ophthalmol. 1929, 12:759-760). Since that time, local anesthetics have been given to millions of patients by the epidural route, intravenously, as stellate ganglion blocks, as peripheral nerve and intercostal nerve blocks, and by nearly every other conceivable route to control the pain of acute zoster and PHN. Once PHN is well established, local anesthetic peripheral nerve, epidural, or synthetic blocks are unlikely to provide more than temporary relief.

There is, therefore, substantial interest in being able to devise simple procedures which will allow for at relatively long term relief from the pain associated with herpes-zoster or PHN.

### SUMMARY OF THE INVENTION

It is accordingly an object of the present invention to provide a method of treating pain in a subject comprising administering to said subject a pharmaceutically effective amount of a porphyrin compound.

The present invention also provides a pharmaceutical composition comprising a porphyrin compound which photosensitizing activity has been altered or modified and a pharmaceutically acceptable carrier.

Other features and advantages of the present invention will become apparent from the following detailed description of the invention.

### BRIEF DESCRIPTION OF THE DRAWINGS

FIGS. 1A-1F show preferred forms of the green porphyrins (formulas 1-6) useful in the methods and compositions of the invention.

FIGS. 2A-2D show preferred forms of hydro-monobenzoporphyrin compounds that are designated as benzoporphyrin derivatives (“BPD's”), namely BPD-DA, BPD-DB, BPD-MA and BPD-MB.

FIG. 3 shows two regioisomers I and II of the green porphyrin compound verteporfin.

### DETAILED DESCRIPTION OF THE INVENTION

In accordance with the invention, it has been surprisingly found that porphyrin compounds are effective in treating pain, in particular neuralgic pain such as the one associated with diabetic neuropathy and post-herpetic neuralgia, more specifically zoster post-herpetic neuralgia.

Porphyrin compounds are very well known for their use in photodynamic therapy of age-related macular degeneration (AMD). Loss of visual acuity is a common problem associated with aging and with various conditions of the eye. Particularly troublesome is the development of unwanted neovascularization in the cornea, retina or choroid. Choroidal neovascularization leads to hemorrhage and fibrosis, with resultant visual loss in a number of recognized eye diseases, including macular degeneration, ocular histoplasmosis syndrome, myopia, and inflammatory diseases. Age-related macular degeneration (AMD) is the leading cause of new blindness in the elderly, and choroidal neovascularization is responsible for 80% of the severe visual loss in patients with this disease. Although the natural history of the disease is eventual quiescence and regression of the neovascularization process, this usually occurs at the cost of sub-retinal fibrosis and vision loss.

Current treatment of AMD includes photodynamic therapy, which relies on low intensity light exposure of photosensitized tissues to produce deleterious effects. Photoactive compounds are administered and allowed to reach a particular undesired tissue which is then irradiated with a light absorbed by the photoactive compound. This results in destruction or impairment of the surrounding tissue.

Photodynamic therapy of conditions in the eye has been attempted over the past several decades using various photoactive compounds, e.g., porphyrin derivatives, such as hematoporphyrin derivative and Photofrin porfimer sodium; “green porphyrins,” such as benzoporphyrin derivative (BPD); and phthalocyanines. Schmidt, U. et al. described experiments using BPD coupled with low density lipoprotein (LDL) for the treatment of Greene melanoma (a nonpigmented tumor) implanted into rabbit eyes and achieved necrosis in this context (IOVS 1992, 33:1253 Abstract 2802). This abstract also describes the success of LDL-BPD in achieving thrombosis in a corneal neovascularization model.

Surprisingly, the porphyrin compounds that normally used in photodynamic therapy were discovered to be effective in pain relief as indicated above.

The mechanism for this effect is not known. However, without being bound by theory, it is believed that one or a combination of the following is responsible for the analgesic activity of porphyrins:

1) The combination of LDL-porphyrin that forms when the drug is administered may be the effector molecule in the dorsal root ganglion of the affected nerve;

2) Porphyrin competes with some local receptor for LDL and in effect lowers the concentration of LDL in the affected neural tissue;

3) Porphyrin has immunomodulatory effects; and/or

4) Porphyrin and/or LDL may have a direct effect on the virus and its cell cycle in the case of post-herpetic neuralgia.

The Porphyrins

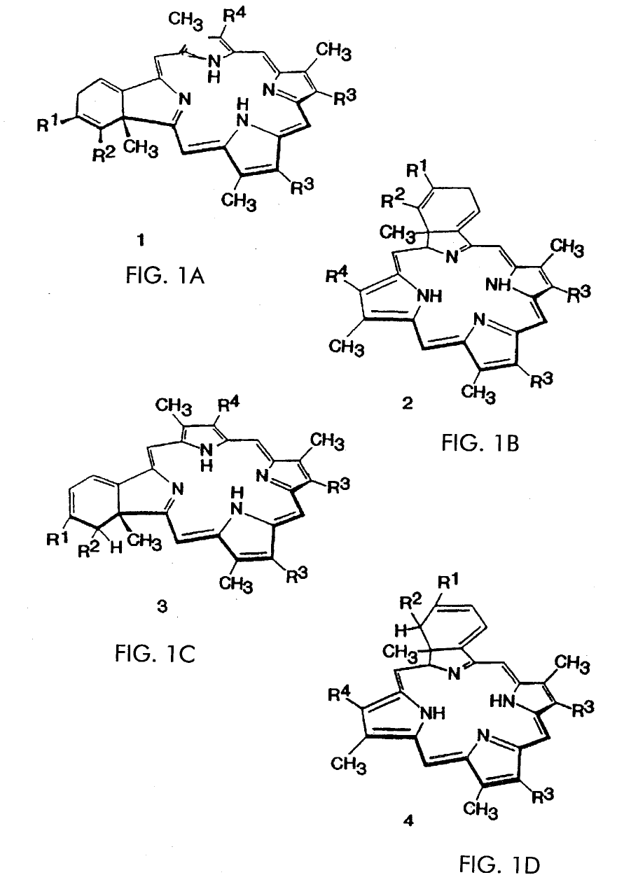

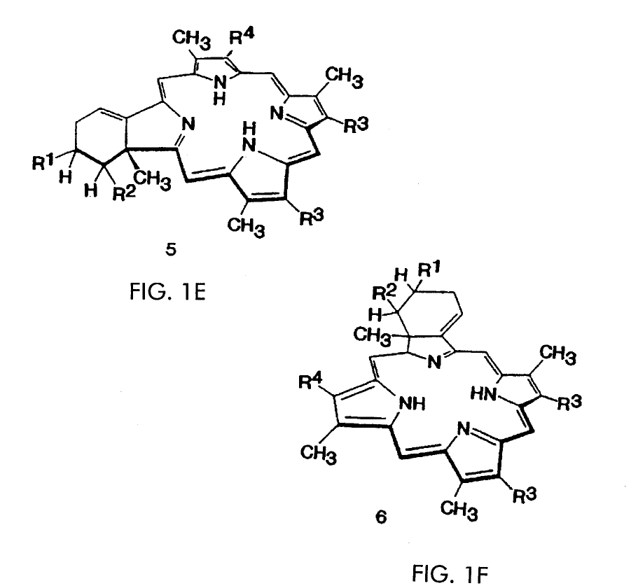

The porphyrins useful in the practice of this invention include the hydro-monobenzoporphyrins (the so-called “green porphyrins” or “Gp” compounds) disclosed in U.S. Pat. Nos. 4,920,143 and 4,883,790. Typically, these compounds have one or more light absorption maxima between about 670-780 nm and are poorly water-soluble (less than 1 mg/ml) or water-insoluble. Gp is preferably selected from the group consisting of those compounds having one of the formulae 1-6 set forth in FIG. 1, mixtures thereof, and the metalated and labeled forms thereof.

In FIG. 1, each R¹ and R² can be independently selected from the group consisting of carbalkoxy (2-6C), alkyl (1-6C) sulfonyl, aryl (6-10C) sulfonyl, aryl (6-10C), cyano, and —CONR⁵CO— wherein R⁵ is aryl (6-10C) or alkyl (1-6C). Preferably, however, each of R¹ and R² is carbalkoxy (2-6C).

Each R³ in FIG. 1 can be independently carboxyalkyl (2-6C) or a salt, amide, ester or acylhydrazone thereof, or is alkyl (1-6C). Preferably R³ is —CH₂—CH₂—COOH or a salt, amide, ester or acylhydrazone thereof.

R⁴ in FIG. 1 can be —CHCH₂; —CHOR^{4′} wherein R^{4′} is H or alkyl (1-6C), optionally substituted with a hydrophilic substituent; —CHO; —COOR^{4′}; —CH(OR^{4′})CH₃; —CH(OR^{4′})CH₂OR^{4′}; —CH(SR⁴)CH₃; —CH(NR^{4′}₂)CH₃; —CH(CN)CH₃; —CH(COOR^{4′})CH₃; —CH(OOCR⁴)CH₃; —CH(halo)CH₃; —CH(halo)CH₂(halo); an organic group of less than 12C resulting from direct or indirect derivatization of a vinyl group; or R⁴ is a 1-3 tetrapyrrole-type nucleus of the formula -L-P, wherein -L- is selected from the group consisting of

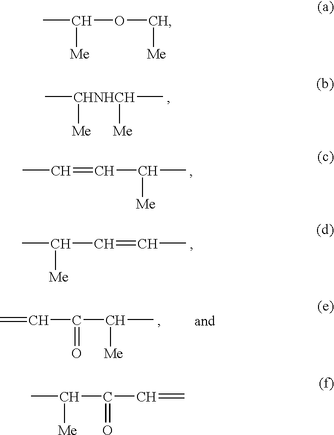

and P is a second Gp, which is one of the formulae 1-6 but lacks R⁴ and is conjugated to L through the position shown as occupied by R⁴, or another porphyrin group. When P is another porphyrin group, P preferably has the formula:

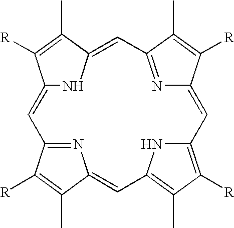

wherein:

each R is independently H or lower alkyl (1-4C); two of the four bonds shown as unoccupied on adjacent rings are joined to R³; one of the remaining bonds shown as unoccupied is joined to R⁴; and the other is joined to L; with the proviso that, if R⁴ is CHCH₂, said R³ groups cannot both be carbalkoxyethyl.

The preparation and use of such compounds are disclosed in U.S. Pat. Nos. 4,920,143 and 4,883,790, which are hereby incorporated by reference.

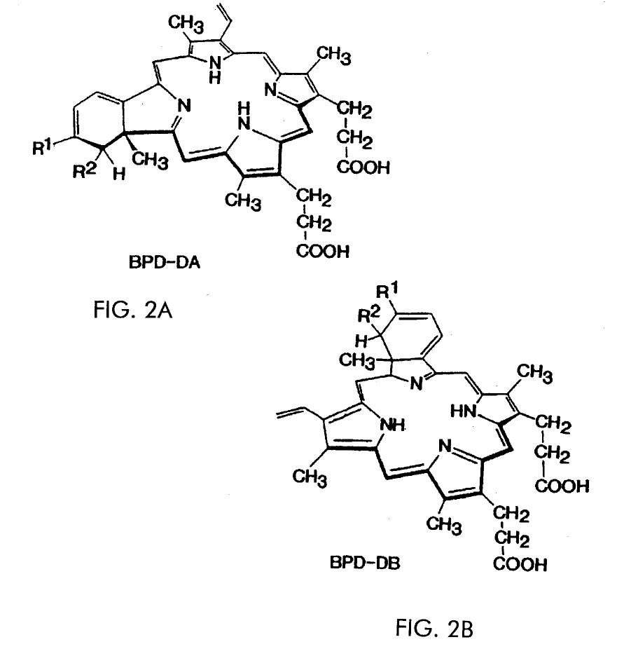

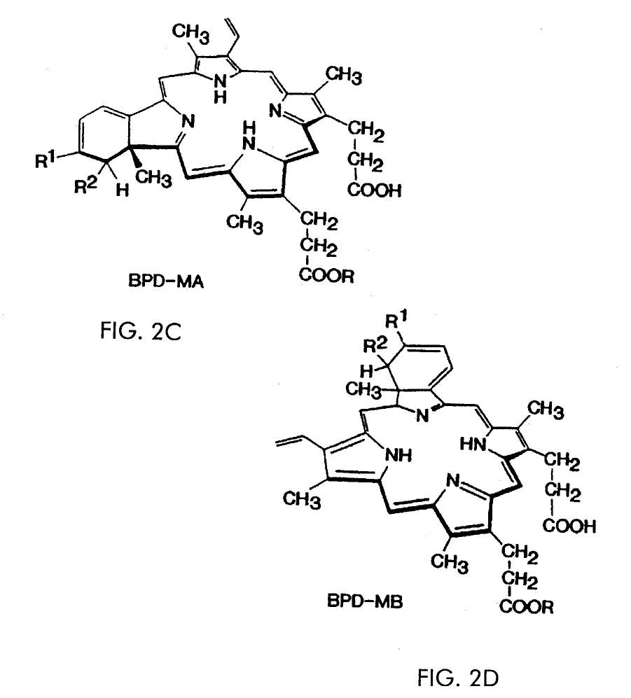

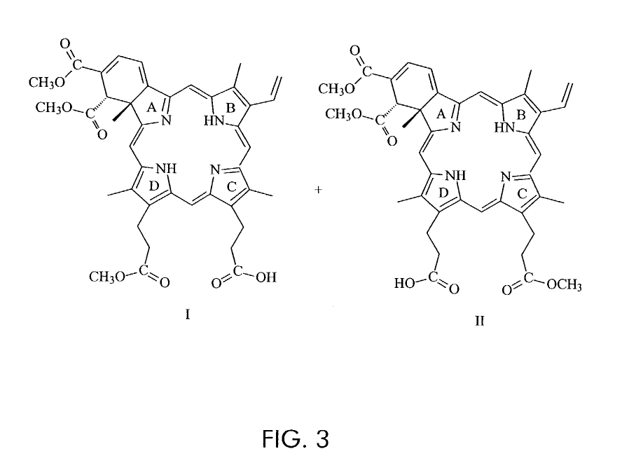

Even more preferred are hydro-monobenzoporphyrin compounds that are designated as benzoporphyrin derivatives (“BPD's”). BPD's are hydrolyzed forms, or partially hydrolyzed forms, of the rearranged products of formula 1-3 or formula 1-4, where one or both of the protected carboxyl groups of R³ are hydrolyzed. Particularly preferred is the compound referred to as BPD-MA in FIG. 2, which has two equally active regioisomers.

Many desirable hydro-monobenzoporphyrin photosensitizers, such as BPD-MA, are not only insoluble in water at physiological pH's, but are also insoluble in (1) pharmaceutically acceptable aqueous-organic co-solvents, (2) aqueous polymeric solutions, and (3) surfactant/micellar solutions. However, such photosensitizers can still be “solubilized” in a form suitable for parenteral administration by using a liposome composition. For example, BPD-MA can be “solubilized” at a concentration of about 2.0 mg/ml in aqueous solution using an appropriate mixture of phospholipids to form encapsulating liposomes.

For a porphyrin compound, photoactivation is the process of generating singlet oxygen which occurs when the compound absorbs a photon of light. This gives an activated porphyrin which transfers its energy to triplet oxygen converting it to the toxic singlet oxygen.

The most preferred porphyrin compound of the present invention is Verteporfin which is a photosensitive dye that is used in the treatment of neovascularization associated with age-related macular degeneration (AMD). Verteporfin is composed of two regioisomers and tightly binds to LDL molecules in the blood stream. It is believed that the drug is selectively partitioned in neovascular tissue because of the high expression of the LDL receptor in this tissue. The dye has a mean half-life of 5-6 hours and is primarily cleared through bile and feces. Less than 1% is cleared via the kidneys.

Verteporfin is currently being administered through intravenous infusion. Following infusion of the dye, a 689 nm laser light (peak absorption) is applied to the macular region of the retina. Verteporfin undergoes a chemical transformation into an excited state that decays back to ground state or to a longer lived lower energy triplet state. This more stable triplet state can interact with oxygen in either a Type I or II reaction. Type I reactions yield hydroxyl radicals, peroxides and superoxides. Type II reactions involve a direct interaction with oxygen to produce singlet oxygen. The Type 11 reactions are thought to play the primary role in causing thrombus formation in neovascular tissue. Other known uses of verteporfin have been in the treatment of psoriasis and skin cancers.

Normally, following administration of porphyrins such as veteporfin, patients are instructed to avoid exposure of skin and eyes to direct sunlight or bright indoor light for about 5 days due to the photosensitivity of these compounds. However, since photoactivation or light treatment of these compounds is apparently not required in order to achieve the desired analgesic effects, it is desirable to modify the photosensitizing activity of porphyrin compounds in order to decrease or avoid this undesirable side effect.

One way to alter the photosensitizing activity of a porphyrin compound is to take away at least one of the conjugated double bonds. Another way is by photobleaching which is the name given to any process which takes place in the presence of light which changes the chromophore (the way in which it absorbs light) of a substance. In general photobleaching is a very complex process and may give rise to a multitude of products. Upon being photobleached, porphyrins such as verteporfin will no longer absorb light at the wavelength at which it is normally activated, thus the photoproducts will no longer be activated at that wavelength.

Formulations

The porphyrin compound is formulated so as to provide an effective concentration to the target tissue. The porphyrin compound may be coupled to a specific binding ligand which may bind to a specific surface component of the target tissue or, if desired, by formulation with a carrier that delivers higher concentrations to the target tissue.

The nature of the formulation will depend in part on the mode of administration and on the nature of the compound selected. Any pharmaceutically acceptable excipient, or combination thereof, appropriate to the particular compound may be used. Thus, the compound may be administered as an aqueous composition, as a transmucosal or transdermal composition, or in an oral formulation. The formulation may also include liposomes. Liposomal compositions are particularly preferred especially where the compound is a green porphyrin. Liposomal formulations are believed to deliver the green porphyrin selectively to the low-density lipoprotein component of plasma which, in turn acts as a carrier to deliver the active ingredient more effectively to the desired site. Increased numbers of LDL receptors have been shown to be associated with locations of neuralgic pain.

Green porphyrins, and in particular BPD-MA, strongly interact with such lipoproteins. LDL itself can be used as a carrier, but LDL is considerably more expensive and less practical than a liposomal formulation. LDLs, or preferably liposomes, are thus preferred carriers for the green porphyrins since green porphyrins strongly interact with lipoproteins and are easily packaged in liposomes. Compositions of green porphyrins involving lipocomplexes, including liposomes, are described in U.S. Pat. No. 5,214,036, U.S. Pat. No. 5,707,608 and U.S. Pat. No. 5,756,541, all of which are being incorporated herein by reference.

Administration and Dosage

The porphyrin compound can be administered in any of a wide variety of ways, for example, orally, parenterally, or rectally. Parenteral administration, such as intravenous, intramuscular, or subcutaneous, is preferred. Intravenous injection is especially preferred.

The dose of the porphyrin compound can vary widely depending on the mode of administration; the formulation in which it is carried, such as in the form of liposomes; or whether it is coupled to a target-specific ligand, such as an antibody or an immunologically active fragment. As is generally recognized, there is a nexus between the type of porphyrin compound, the formulation, the mode of administration, and the dosage level. Adjustment of these parameters to fit a particular combination is possible.

While various porphyrin compounds require different dosage ranges, if green porphyrins are used, a typical dosage is of the range of 0.1-50 mg² (of body surface area) preferably from about 1-10 mg/M² and even more preferably about 2-8 mg/M².

### EXAMPLE 1

A patient presented with age-related macular degeneration and severe pain associated with zoster post-herpetic neuralgia. VISUDYNE® (containing 2 mg of Verteporfin) was used as a photodynamic therapy to treat the patient's neovascular AMD as described above. Briefly, VISUDYNE® was administered through intravenous infusion. Following infusion of the dye, a 689 nm laser light (peak absorption) was applied to the macular region of the retina. Hours after the first infusion, the patient experienced complete resolution of her zoster chronic pain. However, symptoms of pain returned 3 days later.

Thereafter 3 months later and as part of second photodynamic therapy to treat the AMD, the patient received a second dose of VISUDYNE® which again resulted incomplete resolution of neuralgic pain for approximately ten days. However, even after ten days the pain seemed less severe than prior to her initial photodynamic therapy.

### EXAMPLE 2

Patients suffering from pain associated with post-herpetic neuralgia are treated with VISUDYNE® (2 mg of Verteporfin) by administration through intravenous infusion. No light activation is performed following administration. The pain level associated with the post-herpetic neuralgia is significantly reduced after few hours of treatment. Patients in need of follow-up treatments undergo additional treatments days or weeks later as necessary.

### EXAMPLE 3

Patients suffering from pain associated with diabetic neuropathy are treated with VISUDYNE® (2 mg of Verteporfin) by administration through intravenous infusion. No light activation is performed following administration. The pain level associated with the diabetic neuropathy is significantly reduced after few hours of treatment. Patients in need of follow-up treatments undergo additional treatments days or weeks later as necessary.

Although the present invention has been described in relation to particular embodiments thereof, many other variations and modifications and other uses will become apparent to those skilled in the art. The present invention therefore is not limited by the specific disclosure herein, but only by the claims. 00607112.1

What is claimed is:

1. A method of treating pain in a subject comprising administering to said subject a pharmaceutically effective amount of a porphyrin compound.

2. The method of claim 1, wherein said pain is associated with a nerve injury pain or neuralgia.

3. The method of claim 1, wherein said neuralgia is post-herpetic neuralgia.

4. The method of claim 1, wherein said post-herpetic neuralgia is herpes-zoster post-herpetic neuralgia.

5. The method of claim 2, wherein said nerve injury pain is associated with diabetic neuropathy.

6. The method of claim 1, wherein said porphyrin is selected from the group consisting of green porphyrin (hydro-monobenzoporphyrin), hematoporphyrin, and hematoporphyrin derivative.

7. The method of claim 6, wherein said porphyrin is a hydro-monobenzoporphyrin (Gp) compound.

8. The method of claim 7, wherein said green porphyrin is a hydro-monobenzoporphyrin (Gp) compound having a formula selected from the group consisting of: 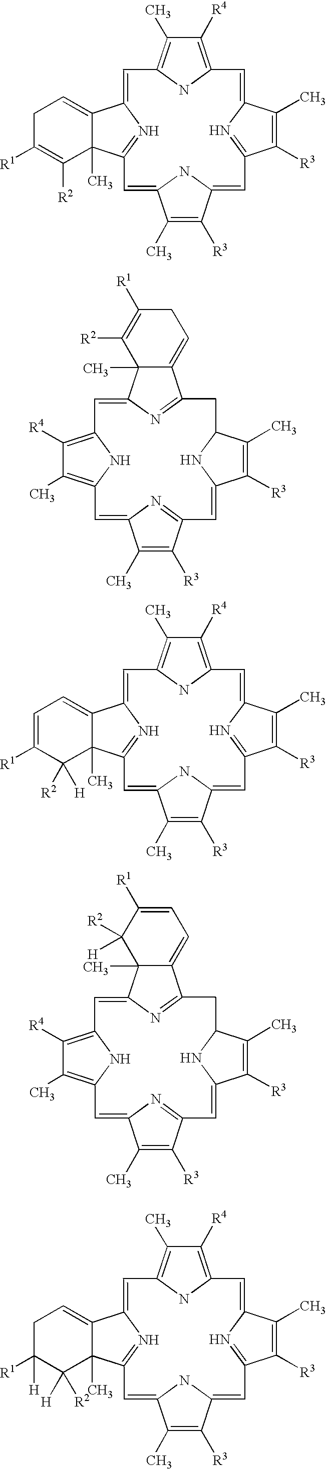
said compound having one or more light absorption maxima between 670-780 nm, or is a metalated or labeled form thereof,
wherein
each R¹ and R² is independently selected from the group consisting of carbalkoxy (2-6C), alkyl (1-6C) sulfonyl, aryl (6-10C) sulfonyl, aryl (6-10C), cyano, and —CONR⁵CO— wherein R⁵ is aryl (6-10C) or alkyl (1-6C);
each R³ is independently carboxyalkyl (2-6C) or a salt, amide, ester or acylhydrazone thereof, or is alkyl (1-6C); and
R⁴ is —CHCH₂; —CHOR^{4′} wherein R^{4′} is H or alkyl (1-6C), optionally substituted with a hydrophilic substituent; —CHO; —COOR^{4′}; —CH(OR^{4′})CH₃; —CH(OR^{4′})CH₂OR^{4′}; —CH(SR⁴)CH₃; —CH(NR^{4′}₂)CH₃; —CH(CN)CH₃; —CH(COOR^{4′})CH₃; —CH(OOCR⁴)CH₃; —CH(halo)CH₃; —CH(halo)CH₂(halo); an organic group of less than 12C resulting from direct or indirect derivatization of a vinyl group; or R⁴ is a 1-3 tetrapyrrole-type nucleus of the formula -L-P, wherein -L- is selected from the group consisting of 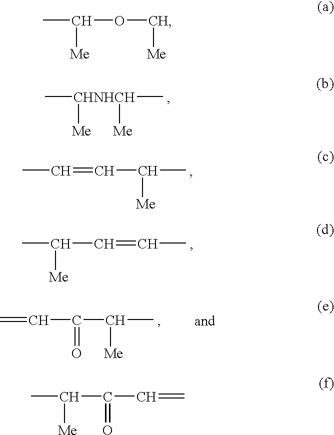
and P is a second Gp, which is one of the formulae 1-6 but lacks R⁴ and is conjugated to L through the position shown as occupied by R⁴, or another porphyrin group.

9. The method of claim 8 wherein, when P is said another porphyrin group, P has the formula: 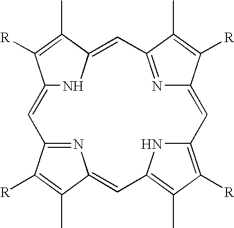
wherein:
each R is independently H or lower alkyl (1-4C);
two of the four bonds shown as unoccupied on adjacent rings are joined to R³;
one of the remaining bonds shown as unoccupied is joined to R⁴; and
the other is joined to L;
with the proviso that, if R⁴ is CHCH₂, said R³ groups cannot both be carbalkoxyethyl.

10. The method of claim 8, wherein each R² is —CH₂—CH₂—COOH or salt, amide, ester or acylhydrazone thereof.

11. The method of claim 8, wherein each of R¹ and R² is carbalkoxy (2-6C).

12. The method of claim 7, wherein said green porphyrin is a hydro-monobenzoporphyrin (Gp) having a formula selected from the group consisting of: 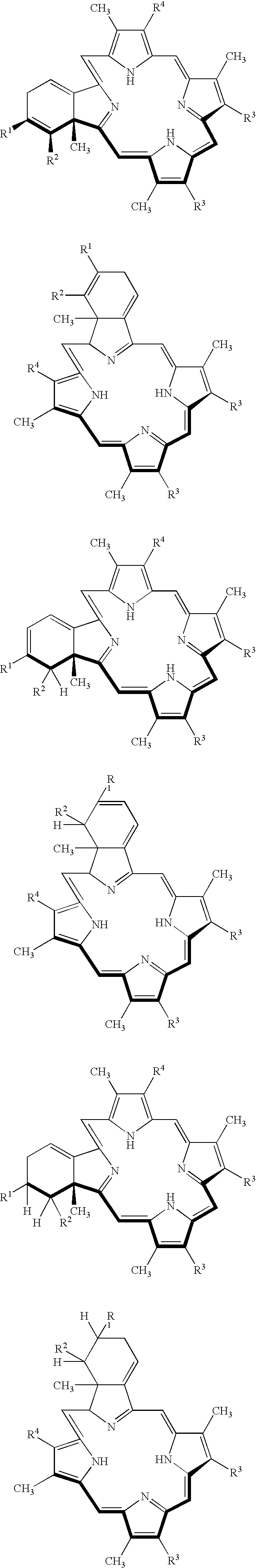
or the metalated and/or labeled form thereof,
wherein each R¹ and R² is independently selected from the group consisting of carbalkoxy (2-6C), alkyl (1-6C) sulfonyl, aryl (6-10C) sulfinyl, aryl (6-10C); cyano; and —CONR⁵CO— where R⁵ is aryl (6-10C) or alkyl (1-6C);
each R³ is independently carboxyalkyl (2-6C) or a salt, amide, ester or acylhydrazone thereof, or is alkyl (1-6C); and
wherein R⁴ is a non-interfering organic group of <12C resulting from direct or indirect derivatization of vinyl.

13. The method of claim 12, wherein R¹ and R² are carbalkoxy.

14. The method of claim 13, wherein R³ and R² are carbomethoxy or carboxethoxy.

15. The method of claim 12, wherein each R³ is —CH₂—CH₂—COOH or a salt, amide, ester or acylhydrazone thereof.

16. The method of claim 13, wherein R³ is —CH₂—CH₂—COOH or a salt, amide, ester or acylhydrazone thereof.

17. The method of claim 1, wherein said compound has a formula selected from group consisting of: 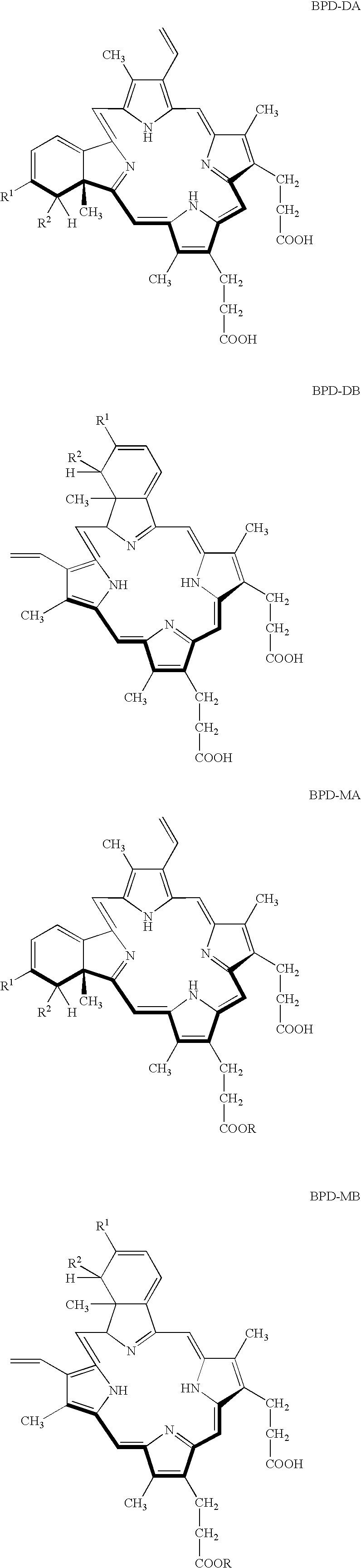
wherein each R¹ and R² is independently selected from the group consisting of carbalkoxy (2-6C), alkyl (1-6C) sulfonyl, aryl (6-10C) sulfinyl, aryl (6-10C); cyano;
and —CONR⁵CO— where R⁵ is aryl (6-10C) or alkyl (1-6C).

18. The method of claim 17, wherein each R¹ and R² is independently alkyl (1-6C).

19. The method of claim 7, wherein said green porphyrin is one of the two following two regioisomers or a combination thereof.

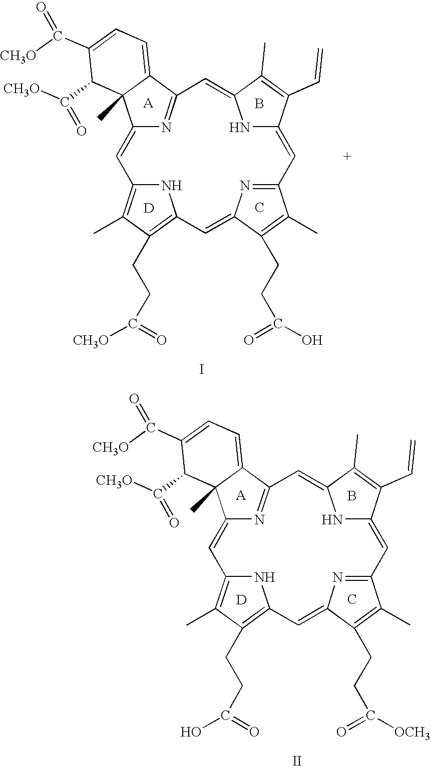

20. The method of claim 19, wherein said green porphyrin is a mixture of the two regioisomers at a ratio of about 21:1.

21. The method of claim 1, wherein said porphyrin is modified to alter or destroy its photosensitizing activity prior to administration.

22. The method of claim 21, wherein said modification is carried out by photobleaching.

23. The method of claim 8, wherein said hydro-monobenzoporphyrin is modified to alter or destroy its photosensitizing activity prior to administration.

24. The method of claim 23, wherein said modification is carried out by photobleaching.

25. The method of claim 23, wherein said modification is carried out by removing at least one conjugate bond from said compound.

26. The method of claim 8, wherein said compound lacks at least one conjugate double bond.

27. A pharmaceutical composition comprising a porphyrin compound which photosensitizing activity has been altered or modified and a pharmaceutically acceptable carrier.

28. The composition of claim 27, wherein said modification is carried out by photobleaching.

29. The composition of claim 27, wherein said modification is carried out by removing at least one conjugate bond from said compound.
